(19) United States
(12) Patent Application Publication
(10) Pub. No.: US 2012/0022151 A1
(43) Pub. Date: Jan. 26, 2012

(54) METHOD FOR PREVENTING DAMAGE TO NUCLEAR MEMBRANE OF SKIN CELL BY ADMINISTERING AMENTOFLAVONE

(75) Inventors: Nok Hyun PARK, Seongnam-si (KR); Yong Joo NA, Yongin-si (KR); Ji Yeong KIM, Seoul (KR); Ji Hyun BAE, Yongin-si (KR); Jun Cheol CHO, Yongin-si (KR)

(73) Assignee: Amorepacific Corporation, Yongsan-gu (KR)

(21) Appl. No.: 13/189,168

(22) Filed: Jul. 22, 2011

(30) Foreign Application Priority Data
Jul. 23, 2010 (KR) 10-2010-0071203

(51) Int. Cl. A61K 31/352; A61P 17/00

(52) U.S. Cl. 514/456

(57) ABSTRACT

Provided are a method for preventing damage to the nuclear membrane of skin cell including administering an effective amount of amentoflavone to a subject and a method for anti-aging including administering an effective amount of amentoflavone to a subject. By controlling the expression of defective lamin A induced by UV radiation or controlling the expression of phosphorylated H2A histone family, member X (H2AX) induced by UV radiation, the disclosed method prevents nuclear membrane damage and thus prevents skin cell damage. Accordingly, a composition containing amentoflavone as an active ingredient may be used as a cosmetic composition or a pharmaceutical composition.

### CROSS-REFERENCE TO RELATED APPLICATION

This application claims priority under 35 U.S.C. §119 to Korean Patent Application No. 10-2010-0071203, filed on Jul. 23, 2010, in the Korean Intellectual Property Office, the disclosure of which is incorporated herein by reference in its entirety. To the extent appropriate, a claim of priority is made to the above disclosed application.

### BACKGROUND 1. Field

The present disclosure relates to a method for preventing damage to skin cell caused by UV.

2. Description of the Related Art

Lamins are proteins that form the fibrillar network in the cell nucleus. Lamins are classified into type A and type B according to the protein structure and gene expression pattern. Lamin A forms lamina on the interior of the nuclear membrane. Mutations in the lamin A gene cause the diseases called laminopathies.

Typical examples are Hutchison-Gilford progeria syndrome (HGPS), atypical Werner's syndrome and mandibuloacral dysplasia (MAD), also known as progeria. Progeria is mostly caused by a point mutation in position 1824 of the gene, replacing cytosine with thymine, creating a form of the lamin gene which cannot be processed properly during expression to protein and accumulates in the cell nucleus.

The representative symptoms of progeria include sclerodermatous skin, hair loss, bone defects, delayed tooth formation, growth retardation, and loss of subcutaneous fat. Abnormalities of the structure and function of cell nucleus are reported in patients with progeria. Nuclear aberration is the representative example.

According to a recent study, nuclear aberration is also observed in elderly people without progeria. The nuclear aberration occurring in old people aged between 81 and 96 is very similar to that of the progeria patients.

Also, increase in the DNA damage marker, phosphorylated H2AX, was found in elderly people, as in the progeria patients.

Accordingly, there is a need of exploring a substance capable of controlling nuclear aberration, expression of defective lamin A and expression of phosphorylated H2AX for anti-aging.

### SUMMARY

The present disclosure is directed to providing a method for preventing damage to the nuclear membrane of skin cell, including administering an effective amount of amentoflavone to a subject.

The method for preventing damage to the nuclear membrane of skin cell may include controlling the expression of defective lamin A induced by UV radiation.

The method for preventing damage to the nuclear membrane of skin cell may include controlling the expression of phosphorylated H2A histone family, member X (H2AX) induced by UV radiation.

In another general aspect, the present disclosure provides a method for anti-aging, including administering an effective amount of amentoflavone to a subject.

The method for anti-aging may include controlling the expression of defective lamin A induced by UV radiation.

The method for anti-aging may include controlling the expression of phosphorylated H2AX induced by UV radiation.

Other features and aspects will be apparent from the following detailed description, the drawings, and the claims.

### BRIEF DESCRIPTION OF THE DRAWINGS

The above and other objects, features and advantages of the present disclosure will become apparent from the following description of certain exemplary embodiments given in conjunction with the accompanying drawings, in which:

FIG. 1 shows damage to the cell nucleus induced by UVB radiation in cells of subjects aged 19 and 39 years;

FIG. 2 shows a western blot result of progerin, a lamin A mutant, and phosphorylated H2AX for a cell nucleus extract from a subject aged 39 years; and

FIG. 3 compares the degree of nuclear damage and expression of phosphorylated H2AX caused by lamin A defect in cells of a subject aged 39 years, in the UVB-treated group and the amentoflavone-treated group.

### DETAILED DESCRIPTION

The advantages, features and aspects of the present disclosure will become apparent from the following description of the embodiments with reference to the accompanying drawings, which is set forth hereinafter. The present disclosure may, however, be embodied in different forms and should not be construed as limited to the embodiments set forth herein. Rather, these embodiments are provided so that this disclosure will be thorough and complete, and will fully convey the scope of the present disclosure to those skilled in the art. The terminology used herein is for the purpose of describing particular embodiments only and is not intended to be limiting of the example embodiments. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof.

Hereinafter, exemplary embodiments will be described in detail with reference to the accompanying drawings.

Amentoflavone is represented by Chemical Formula 1:

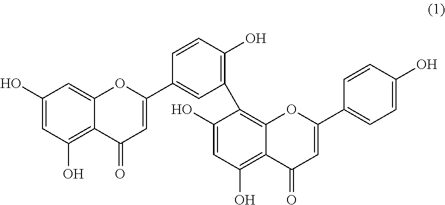

The inventors of the present disclosure have found out that treatment of fibroblasts with UV leads to increased expression of defective lamin A and structural aberration of the nuclear membrane. Also, the expression of phosphorylated H2A histone family, member X (H2AX, NCBI ref. seq. NP_{—}002096.1) was increased. They have also found out that treatment with amentoflavone reduced the expression of defective lamin A and retained the structure of the nuclear membrane. Also, the expression of the DNA damage marker, phosphorylated H2AX, decreased by the treatment with amentoflavone.

The UV may be UVA, UVB or UVC.

The present disclosure provides a method for anti-aging, comprising administering an effective amount of amentoflavone to a subject. The method for anti-aging may comprise administering amentoflavone to a subject in the form of a cosmetic composition or a pharmaceutical composition.

The present disclosure also provides a cosmetic composition or a pharmaceutical composition for preventing damage to skin cell caused by UV comprising amentoflavone as an active ingredient.

The pharmaceutical composition for preventing damage to skin cell may comprise amentoflavone as well as a pharmaceutically acceptable carrier, excipient or diluent.

The pharmaceutical composition of the present disclosure may be prepared into formulations for oral administration such as powder, granule, tablet, capsule, suspension, emulsion, syrup, aerosol, etc., or formulations for parenteral administration such as topical solution, suppository, sterile injection, etc. according to methods known in the art. The carrier, excipient or diluent that may be included in the pharmaceutical composition of the present disclosure may be, for example, lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate or mineral oil. Also, commonly used filler, thickener, binder, wetting agent, disintegrant, surfactant, etc. may be used as the diluent or excipient. Solid preparations for oral administration include tablet, pill, powder, granule, capsule, or the like. The solid preparation may be prepared by adding one or more excipient, e.g. starch, calcium carbonate, sucrose, lactose, gelatin, etc., to the composition of the present disclosure. In addition to simple excipient, a lubricant such as magnesium stearate or talc may be added. Liquid preparations for oral administration include suspension, internal solution, emulsion, syrup, or the like. They may include, in addition to simple diluent such as water or liquid paraffin, various other excipients such as wetting agent, sweetener, fragrance, antiseptic, or the like. Preparations for parenteral administration include sterile aqueous solution, non-aqueous solution, suspension, emulsion, lyophilized preparation and suppository. For the non-aqueous solution or suspension, propylene glycol, polyethylene glycol, vegetable oil such as olive oil, injectable ester such as ethyl oleate, or the like may be used. As a base of the suppository, witepsol, macrogol, Tween 61, cocoa butter, laurin butter, glycerogelatin, etc. may be used.

The composition of the present disclosure may be administered orally or parenterally (e.g., intravenously, subcutaneously, intraabdominally or topically) depending on purposes. The administration dosage may be determined variously depending on the condition, body weight, age and sex of the patient, diet, excretion rate, severity of disease, composition type, administration time, administration method, administration route, administration period, or the like. A daily administration dosage is 100-300 mg/kg, specifically 100-200 mg/kg based on weight of the lyophilized preparation. As occasion demands, the composition may be administered once or several times a day.

The examples and experiments will now be described. The following examples and experiments are for illustrative purposes only and not intended to limit the scope of this disclosure.

### EXAMPLE 1

### Expression of Lamin A Induced By UVB Radiation in Subjects Aged 19 and 39

Cells of subjects aged 19 or 39 years were distributed in a 4-well chamber slide holding a medium prepared by adding 1% LSGS and 100 IU penicillin G to 106 medium at 30,000 cells/well. The cells were divided into a UVB-untreated group and a UVB-treated group.

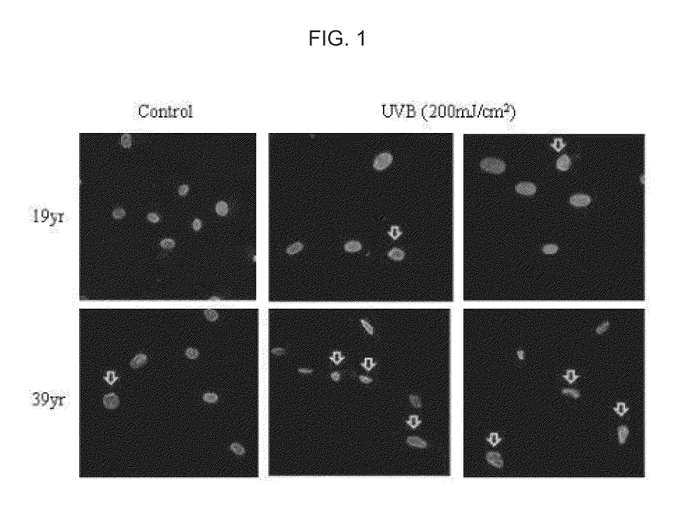

The next day, the medium was replaced with 106 medium containing 1% fetal bovine serum (FBS). 24 hours later, after washing the well with PBS, UVB was irradiated at 200 mJ/cm² only to the UVB-treated group. Then, after treating again with 106 medium containing 1% FBS, the cells were cultured at 37° C. for 6 days. The treated cells were subjected to immunofluorescence (IF) staining. After washing the well with PBS containing 1 mM CaCl₂ and 1 mM MgCl₂, the cells were fixed at room temperature for 10 minutes in 3.5% paraformaldehyde solution. The fixed cells were washed 3 times for 10 minutes with PBS, treated with 0.1% TritonX-100 for 5 minutes, and then washed 3 times for 10 minutes with PBS. After reaction at 4° C. O/N with lamin A antibody (Santa Cruz) diluted to 1:300 with PBS containing 0.05% Tween 20 (PBST) as primary antibody, the cells were washed 3 times for 10 minutes with PBST and then reacted at room temperature for 1 hour with FITC-conjugated secondary antibody (1:400). Then, after washing 3 times for 10 minutes with PBST, a mounting solution was added and a cover glass was laid. The stained cells were imaged by confocal laser scanning microscopy (Ziess). The result is shown in FIG. 1. More damage to the cell nucleus was observed in the cells from the 39-year-old subject than those from the. 19-year-old subject.

### EXAMPLE 2

### Expression of Mutant Lamin A Induced By UVB Radiation in Subjects Aged 19 and 39 and Inhibitory Effect Thereof By Amentoflavone

Cells of subjects aged 19 or 39 years were distributed in a 4-well chamber slide holding a medium prepared by adding 1% LSGS and 100 IU penicillin G to 106 medium at 100,000 cells/well. The cells were divided into a UVB-untreated negative control group, a UVB-treated group and a UVB/amentoflavone-treated group. The next day, the medium was replaced with 106 medium containing 1%

FBS. The amentoflavone-treated group was treated with 5 μM amentoflavone prior to UVB radiation. 24 hours later, after washing the well with PBS, UVB was irradiated at 100 mJ/cm² to the test groups. Then, after treating again with 106 medium containing 1% FBS, the cells were cultured at 37° C. for 6 days. The treated cells were subjected to western blot and immunofluorescence staining. For western blot, the cells were lysed using RIPA buffer (Sigma) and proteins were quantitated according to the Lowry's method. 10 μg of each protein was run on 4-12% Nupage gel (Invitrogen) and then transferred to NC membrane. The membrane was blocked with 5% BSA and reacted O/N at 4° C. with the lamin A mutant antibodies, progerin (Abcam) and p-H2AX (Millipore), diluted at 1:300 and 1:200, respectively, with 5% BSA. The next day, the membranes were washed 3 times for 10 minutes with TBS containing 0.05% Tween 20 (TBST) and then reacted at room temperature for 1 hour with HRP-conjugated mouse and rabbit secondary antibodies (1:1000). Then, after washing 3 times for 10 minutes with PBST, bands were detected using ECL and LAS3000.

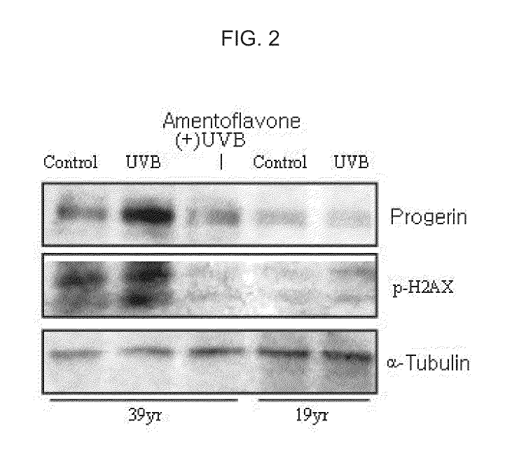

The result is shown in FIG. 2. UVB radiation resulted in increase of progerin (mutant lamin A), which was decreased by the treatment with amentoflavone. The same result was shown for phosphorylated H2AX. Tubulin was used as protein loading control.

For immunofluorescence staining, after washing the well with PBS containing 1 mM CaCl₂ and 1. mM MgCl₂, the cells were fixed at room temperature for 10 minutes in 3.5% paraformaldehyde solution. The fixed cells were washed 3 times for 10 minutes with PBS, treated with 0.1% TritonX-100 for 5 minutes, and then washed 3 times for 10 minutes with PBS. After reaction at 4° C. O/N with lamin A antibody (Santa. Cruz) diluted to 1:300 or p-H2AX antibody (Millipore) diluted to 1:200 with PBS containing 0.05% Tween 20 (PBST) as primary antibodies, the cells were washed 3 times for 10 minutes with PBST and then reacted at room temperature for 1 hour with FITC- and rhodamine-conjugated secondary antibody (1:400). Then, after washing 3 times for 10 minutes with PBST and staining the nucleus with propidium iodide (PI), a mounting solution was added and a cover glass was laid. The stained cells were imaged by confocal laser scanning microscopy (Ziess).

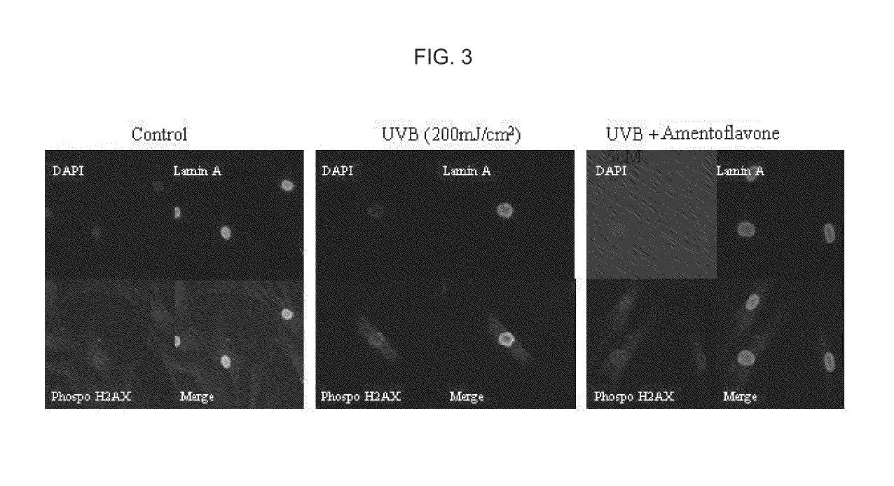

The result is shown in FIG. 3. UVB radiation resulted in increased nuclear damage and increased expression of phosphorylated H2AX caused by lamin A defects in the cells of the 39-year-old subject. The treatment with amentoflavone reduced the expression of phosphorylated H2AX and retained the structure of the cell nucleus.

The preparation examples of the cosmetic composition or pharmaceutical composition of the present disclosure will now be described. The following examples are for illustrative purposes only and not intended to limit the scope of this disclosure.

### PREPARATION EXAMPLE 1

### Softening Lotion (Skin Lotion)

Softening lotion was prepared according to a commonly employed method as described in Table 1.

TABLE 1

| Ingredients | Contents (weight %) |
|---|---|
| Amentoflavone | 2.0 |
| Glycerin | 3.5 |
| Oleyl alcohol | 1.5 |
| Ethanol | 5.5 |
| Polysorbate 80 | 3.2 |
| Carboxyvinyl polymer | 1.0 |
| Butylene glycol | 2.0 |
| Propylene glycol | 2.0 |
| Antiseptic & fragrance | adequate |
| Purified water | balance |
| Total | 100 |

### PREPARATION EXAMPLE 2

### Nourishing Lotion (Milk Lotion)

Nourishing lotion was prepared according to a commonly employed method as described in Table 2.

TABLE 2

| Ingredients | Contents (weight %) |
|---|---|
| Amentoflavone | 2.0 |
| Glycerin | 3.0 |
| Butylene glycol | 3.0 |
| Propylene glycol | 3.0 |
| Carboxyvinyl polymer | 0.1 |
| Beeswax | 4.0 |
| Polysorbate 60 | 1.5 |
| Caprylic/capric triglyceride | 5.0 |
| Squalane | 5.0 |
| Sorbitan sesquiolate | 1.5 |
| Cetearyl alcohol | 1.0 |
| Triethylamine | 0.2 |
| Antiseptic & fragrance | adequate |
| Purified water | balance |
| Total | 100 |

### PREPARATION EXAMPLE 3

### Nourishing Cream

Nourishing cream was prepared according to a commonly employed method as described in Table 3.

TABLE 3

| Ingredients | Contents (weight %) |
|---|---|
| Amentoflavone | 2.0 |
| Glycerin | 3.5 |
| Butylene glycol | 3.0 |
| Liquid paraffin | 7.0 |
| Beta-glucan | 7.0 |
| Carbomer | 0.1 |
| Caprylic/capric triglyceride | 3.0 |
| Squalane | 5.0 |
| Cetearyl glucoside | 1.5 |
| Sorbitan stearate | 0.4 |
| Polysorbate 60 | 1.2 |
| Triethylamine | 0.1 |
| Antiseptic & fragrance | adequate |
| Purified water | balance |
| Total | 100 |

### PREPARATION EXAMPLE 4

### Massage Cream

Massage cream was prepared according to a commonly employed method as described in Table 4.

TABLE 4

| Ingredients | Contents (weight %) |
|---|---|
| Amentoflavone | 2.0 |
| Glycerin | 8.0 |
| Butylene glycol | 3.0 |
| Liquid paraffin | 45.0 |
| Beta-glucan | 7.0 |
| Carbomer | 0.1 |
| Caprylic/capric triglyceride | 3.0 |
| Beeswax | 4.0 |
| Cetearyl glucoside | 1.5 |
| Sorbitan sesquiolate | 0.9 |
| Paraffin | 1.5 |
| Antiseptic, pigment & fragrance | adequate |
| Purified water | balance |
| Total | 100 |

### PREPARATION EXAMPLE 5

### Pack

Pack was prepared according to a commonly employed method as described in Table 5.

TABLE 5

| Ingredients | Contents (weight %) |
|---|---|
| Amentoflavone | 2.0 |
| Glycerin | 4.0 |
| Polyvinyl alcohol | 15.0 |
| Hyaluronic acid | 5.0 |
| Beta-glucan | 7.0 |
| Allantoin | 0.1 |
| Nonyl phenyl ether | 0.4 |
| Polysorbate 60 | 1.2 |
| Ethanol | adequate |
| Antiseptic & fragrance | adequate |
| Purified water | balance |
| Total | 100 |

### PREPARATION EXAMPLE 6

### Ointment for Topical Skin Application

Ointment was prepared according to a commonly employed method as described in Table 6.

TABLE 6

| Ingredients | Contents (weight %) |
|---|---|
| Amentoflavone | 2.0 |
| Beta-1,3-glucan | 10.0 |
| Beeswax | 10.0 |
| Polysorbate | 5.0 |
| PEG 60 hydrogenated castor oil | 2.0 |
| Sorbitan sesquiolate | 0.5 |
| Vaseline | 5.0 |
| Liquid paraffin | 10.0 |
| Squalane | 5.0 |
| Shea butter | 3.0 |
| Caprylic/capric triglyceride | 5.0 |
| Glycerin | 10.0 |
| propylene glycol | 10.2 |
| Triethylamine | 0.2 |
| Antiseptic, pigment & fragrance | adequate |
| Purified water | balance |
| Total | 100 |

### PREPARATION EXAMPLE 7

### Patch for Topical Administration

Patch for topical administration was prepared according to a commonly employed method as described in Table 7.

TABLE 7

| Ingredients | Contents (weight %) |
|---|---|
| Amentoflavone | 2.0 |
| Beta-1,3-glucan | 3.0 |
| Diethylamine | 0.7 |
| Sodium sulfite | 0.1 |
| Polyoxyethylene lauryl ether (EO = 9) | 1.0 |
| Polyhydroxyethylene cetyl stearyl ether | 1.0 |
| (Cetomacrogol 1000) | |
| Viscous paraffin oil | 2.5 |
| Caprylic/capric acid ester (Cetiol LC) | 2.5 |
| Polyethylene glycol 400 | 3.0 |
| Polyacrylic acid (Carbopol 934P) | 1.0 |
| Purified water | balance |
| Total | 100 |

### PREPARATION EXAMPLE 8

### Preparation of Powder

| | |
|---|---|
| Amentoflavone | 2 g |
| Lactose | 1 g |

The above ingredients were mixed and filled in an airtight pouch.

### PREPARATION EXAMPLE 9

### Preparation of Tablet

| | |
|---|---|
| Amentoflavone | 100 mg |
| Corn starch | 100 mg |
| Lactose | 100 mg |
| Magnesium stearate | 2 mg |

The above ingredients were mixed and prepared into tablet according to a commonly employed method.

### PREPARATION EXAMPLE 10

### Preparation of Capsule

| | |
|---|---|
| Amentoflavone | 100 mg |
| Corn starch | 100 mg |
| Lactose | 100 mg |
| Magnesium stearate | 2 mg |

The above ingredients were mixed and filled in a gelatin capsule according to a commonly employed method.

### PREPARATION EXAMPLE 11

### Preparation of Pill

| | |
|---|---|
| Amentoflavone | 1 g |
| Lactose | 1.5 g |
| Glycerin | 1 g |
| Xylitol | 0.5 g |

The above ingredients were mixed and prepared into pill weighing 4 g each according to a commonly employed method.

### PREPARATION EXAMPLE 12

### Preparation of Granule

| | |
|---|---|
| Amentoflavone | 150 g |
| Soybean extract | 50 mg |
| Glucose | 200 mg |
| Starch | 600 mg |

The above ingredients were mixed and, after adding 100 mg of 30% ethanol and drying at 60° C., the resulting granule was filled in a pouch.

### PREPARATION EXAMPLE 13

### Preparation of Injection

| | |
|---|---|
| Amentoflavone | 10 mg |
| Mannitol | 180 mg |
| Sterile distilled water for injection | 2974 mg |
| Na₂HPO₄•12H₂O | 26 mg |

The above ingredients were mixed and filled in a 2-mL ampule according to a commonly employed method.

### PREPARATION EXAMPLE 14

### Preparation of Solution

| | |
|---|---|
| Amentoflavone | 20 mg |
| Isomerized glucose | 10 g |
| Mannitol | 5 g |
| Purified water | adequate |

The above ingredients were dissolved in purified water. After adding lemon flavor and mixing the ingredients, purified water was added to make 100 mL. The resulting solution was filled in a brown bottle.

By controlling the expression of defective lamin A induced by UV radiation or controlling the expression of phosphorylated H2A histone family, member X (H2AX) induced by UV radiation, the disclosed method prevents nuclear membrane damage and thus prevents skin cell damage. Accordingly, a composition containing amentoflavone as an active ingredient may be used as a Cosmetic composition or a pharmaceutical composition.

While the present disclosure has been described with respect to the specific embodiments, it will be apparent to those skilled in the art that various changes and modifications may be made without departing from the spirit and scope of the disclosure as defined in the following claims.

What is claimed is:

1. A method for preventing damage to the nuclear membrane of skin cell, comprising administering an effective amount of amentoflavone to a subject.

2. The method for preventing damage to the nuclear membrane of skin cell according to claim 1, which comprises controlling the expression of defective lamin A induced by UV radiation.

3. The method for preventing damage to the nuclear membrane of skin cell according to claim 1, which comprises controlling the expression of phosphorylated H2A histone family, member X (H2AX) induced by UV radiation.

4. A method for anti-aging, comprising administering an effective amount of amentoflavone to a subject.

5. The method for anti-aging according to claim 4, which comprises controlling the expression of defective lamin A induced by UV radiation.

6. The method for anti-aging according to claim 4, which comprises controlling the expression of phosphorylated H2A histone family, member X (H2AX) induced by UV radiation.
